(19) United States
(12) Patent Application Publication
(10) Pub. No.: US 2026/0341431 A1
(43) Pub. Date: Oct. 29, 2026

(54) MACHINE LEARNING FOR PATIENT INTAKE AND CLAIM APPROVAL PREDICTION

(71) Applicant: MatrixCare, Inc., Bloomington, MN (US)

(72) Inventors: Dustin Hans Willy FELD, Köln (DE); Vivek KUMAR, Windermere, FL (US); Melissa KOZAK, Millwood, NY (US)

(21) Appl. No.: 19/632,727

(22) Filed: Mar. 30, 2026

Related U.S. Application Data
(60) Provisional application No. 63/793,577, filed on Apr. 23, 2025.

(51) Int. Cl. G16H 10/60; G16H 50/20

(52) U.S. Cl. CPC G16H 10/60; G16H 50/20

(57) ABSTRACT

Techniques for improved machine learning are provided. An approval request specifying a plurality of values for a plurality of request fields is accessed. A model output is generated based on processing the plurality of values using a machine learning model, wherein the output comprises an approval prediction for the approval request, and a denial reason for the approval request. The model output is outputted, and an updated value for at least one of the plurality of values is received. A second model output is generated based on processing the updated value using the machine learning model, and the second model output is outputted.

### CROSS-REFERENCE TO RELATED APPLICATIONS

This application claims benefit of co-pending U.S. provisional patent application Ser. No. 63/793,577 filed Apr. 23, 2025. The aforementioned related patent application is herein incorporated by reference in its entirety.

### TECHNICAL FIELD

The present disclosure relates generally to machine learning, and more particularly, to use of machine learning to facilitate patient intake and/or predict claim approval.

A wide variety of care settings and facilities are often available to treat patients. The best facility to treat a given patient may vary over time, and patients often transfer between multiple facilities. Patient intake to a new facility is an intensive and error-prone process. For example, a vast assortment of useful information may be lost during the intake process, resulting in wasted time and expense (e.g., to re-collect the data) and, in some cases, sub-optimal therapeutic outcomes. Additionally, in many care settings, approval may be needed or desired for a variety of services or actions. However, the process of requesting such approval is opaque and complex, resulting in substantial wasted computational resources in repeatedly re-generating and transmitting requests when previous requests are denied.

Improved systems and techniques to leverage machine learning for intake and/or claim processing are desired.

### SUMMARY

According to some implementations of the present disclosure, a method includes: accessing a first approval request specifying a first plurality of values for a plurality of request fields; generating a first model output based on processing the plurality of values using a machine learning model, wherein the first output comprises: a first approval prediction for the first approval request, and a first denial reason for the first approval request; outputting the first model output; receiving an updated value for at least one of the first plurality of values; generating a second model output based on processing the updated value using the machine learning model; and outputting the second model output.

According to some implementations of the present disclosure, a method includes: receiving a plurality of documents comprising medical data for an individual; extracting the medical data from the plurality of documents using an automated document ingestion operation; generating a patient record for the individual based on the medical data; generating one or more risk predictions based on processing the patient record using one or more risk prediction machine learning models; generating a care plan for the individual based on processing the patient record and the one or more risk predictions using one or more care plan machine learning models; and generating an intake patient record based on the patient record and the care plan.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective embodiments.

FIG. 1 depicts an example system for machine learning-based approval predictions, according to some embodiments of the present disclosure.

FIG. 2 depicts an example workflow for using machine learning models to predict approval and refine requests, according to some embodiments of the present disclosure.

FIG. 3 depicts an example workflow for training machine learning models to predict approval and refine requests, according to some embodiments of the present disclosure.

FIG. 4 depicts an example system for machine learning-based patient intake, according to some embodiments of the present disclosure.

FIG. 5 depicts an example workflow for using machine learning models to facilitate patient intake, according to some embodiments of the present disclosure.

FIG. 6 depicts an example graphical user interface for machine learning-based patient intake, according to some embodiments of the present disclosure.

FIG. 7 is a flow diagram depicting an example method for using machine learning to evaluate approval requests, according to some embodiments of the present disclosure.

FIG. 8 is a flow diagram depicting an example method for training machine learning models to evaluate approval requests, according to some embodiments of the present disclosure.

FIG. 9 is a flow diagram depicting an example method for on-line refinement of machine learning models to evaluate approval requests, according to some embodiments of the present disclosure.

FIG. 10 is a flow diagram depicting an example method for using machine learning to improve patient intake, according to some embodiments of the present disclosure.

FIG. 11 is a flow diagram depicting an example method evaluating approval requests, according to some embodiments of the present disclosure.

FIG. 12 is a flow diagram depicting an example method patient intake, according to some embodiments of the present disclosure.

FIG. 13 depicts an example computing device configured to perform various request evaluation aspects of the present disclosure, according to some embodiments disclosed herein.

FIG. 14 depicts an example computing device configured to perform various patient intake aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for using machine learning for facilitating patient intake and/or evaluating and generating approval requests.

In some embodiments, machine learning models can be trained to predict the probability that an approval request will be approved. For example, in the context of healthcare, one may submit requests for approval prior to providing items (e.g., medication) and/or performing actions (e.g., operations), and/or for reimbursement after providing the items and/or performing the action. Often, these approval requests include substantial contextual detail that the respondent uses to determine whether to approve or reject the requests. However, the actual processes used to evaluate such requests are opaque and seemingly contradictory at times. In some embodiments, therefore, machine learning models can be trained to predict whether a given request will be approved, prior to actually submitting the request.

This can reduce the computational expense incurred by the computing systems. For example, requesting systems may otherwise generate and transmit requests repeatedly (e.g., modifying the request slightly before re-transmitting it) until approval is received. However, this wastes substantial computing resources of the requesting system (e.g., generating many requests with no guidance as to which, if any, will be approved). Further, the repeated transmission of such requests consumes substantial network bandwidth, potentially impacting other traffic on the network. Similarly, from the perspective of the responding systems, substantial computational expense is wasted evaluating and denying the repeated malformed or inadequate requests (e.g., evaluating multiple requests for the same action). Advantageously, by using machine learning to evaluate such requests prior to transmission, acceptance probability can be increased, which reduces computational expense.

Further, in some embodiments, machine learning model(s) may be trained and/or used to predict the most probable reasons for denial of a request. Advantageously, this insight may facilitate more rapid correction of any deficiencies in the request, resulting not only in increased approval probability but also reduced computational expense (e.g., which is often incurred by repeatedly modifying and re-transmitting the request until approval).

In some embodiments, machine learning models can be used to facilitate improved patient intake (in conjunction with facilitating request evaluation, or as a standalone workflow). For example, to perform intake of a patient to a facility, it is often desirable to ingest the patient's relevant medical data. However, most healthcare facilities utilize discrete medical data systems (often using different formatting, structures, and the like), and direct data sharing between these systems is at best complex and in many cases, intractable. In many cases, therefore, the intake facility that is receiving the patient also receives a voluminous set of documentation including medical data for the patient. However, this medical data is rarely (or never) in a digital format. Instead, most of the documentation is provided as hard copy (e.g., physical printouts of the patient's information) and/or via fax machine (e.g., where physical documents are scanned prior to being faxed).

In some embodiments, although this documentation contains useful medical information, the information is generally unformatted (or formatted according to an unknown format), and often includes duplicative and/or outdated details. Further, in many cases, some or all of the documents are entirely unstructured (e.g., freeform natural language text).

In some embodiments, machine learning models can be used to facilitate the intake process. In some embodiments, raw documentation can be accessed and processed using one or more operations (e.g., optical character recognition (OCR)) to extract the text therein. In some embodiments, this text can be evaluated using various models to identify the content and/or context of each document, extract relevant medical data, and the like, as discussed in more detail below. This medical information can then be used to automatically generate a patient record.

In some embodiments, this patient record can then be processed using various machine learning models to evaluate or predict various risks that are (or may be) relevant for the patient, such as the risk that the patient will suffer a fall, the risk that the patient will require hospitalization, the risk that the patient will suffer one or more specified disorders or diseases, and the like. In some embodiments, these risk predictions can be processed, along with the patient record itself, using additional machine learning models to generate and/or evaluate care plans for the patient (e.g., suggesting a set of support or healthcare-related actions for the patient).

In some embodiments, this information (e.g., the patient record, the predicted risks, and/or the generated care plan) can then be processed using further machine learning models to generate a selection of the care facility where the patient should be treated. That is, the system may predict which specific facility, of a set of possible alternatives, should receive the patient based on the generated information. In some embodiments, the patient can then facilitate this intake, such as by generating a unified intake record including all of the relevant data (e.g., the data in the patient record extracted from the received documentation, the predicted risks, the suggested care plans, the suggested facilities, and the like). This intake record can then be used to intake the patient to the indicated facility, to treat the patient in accordance with the generated data, and/or to discharge the patient (immediately or when appropriate, based on the predictions).

Advantageously, this automated intake process can significantly reduce the potential for error, as well as improving patient outcomes. For example, some conventional approaches assume that the received data is formatted appropriately for intake, but these systems fail to operate effectively when the formatting changes. As another example, some conventional systems use simplistic extraction methods to determine medical data without further processing (e.g., de-duplication, identifying outdated information, and the like), similarly resulting in sub-optimal results. By utilizing some embodiments of the present disclosure, the intake process can be accelerated and computational expense spent ingesting documents can be reduced.

### Example System for Machine Learning-Based Approval Predictions

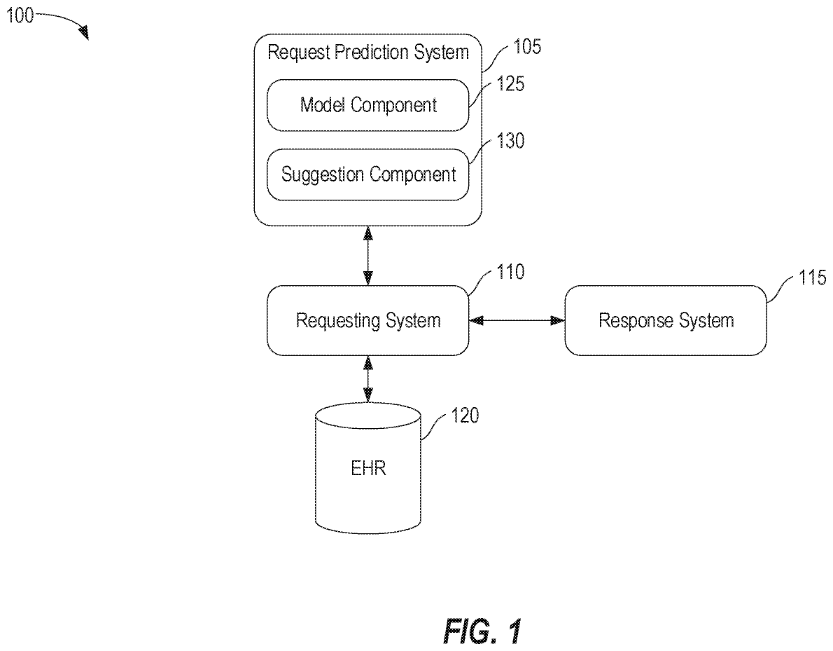

FIG. 1 depicts an example system 100 for machine learning-based approval predictions, according to some embodiments of the present disclosure.

In the illustrated example, the system 100 includes a request prediction system 105, a requesting system 110, a response system 115, and a repository of electronic health records (EHR) 120. Although depicted as discrete systems for conceptual clarity, the operations of the request prediction system 105, the requesting system 110, and/or the response system 115 may be combined or distributed across any number and variety of computing systems. Further, the operations of the depicted systems may generally be implemented using hardware, software, or a combination of hardware and software. Additionally, although a single repository of EHR 120 is depicted for conceptual clarity, in some embodiments, there may be any number of sources of the EHR 120.

In the illustrated example, the various components of the system 100 are communicatively coupled via one or more communication links, which may include any number and variety of wired connections and/or wireless connections. In some embodiments, some or all of the components communicate via one or more networks, such as the Internet. In the illustrated system 100, the request prediction system 105 is communicatively coupled with the requesting system 110. Although not depicted in the illustrated example, in some embodiments, the request prediction system 105 may additionally or alternatively be communicatively linked to the EHR 120 and/or the response system 115. Further, in the depicted example, the requesting system 110 is communicatively coupled with the request prediction system 105, the EHR 120, and the response system 115.

The requesting system 110 is generally representative of a computing system used to generate, revise, review, evaluate, and/or transmit approval requests. For example, the requesting system 110 may correspond to a computing device of a healthcare facility (e.g., on the premises, or otherwise associated with the facility). In some embodiments, some or all of the approval requests can be generated based, at least in part, on the EHR 120. The EHR 120 generally includes medical and/or healthcare-related information for patients associated with the healthcare facility to which the requesting system 110 corresponds. For example, for any given patient, the EHR 120 may include patient details such as demographic information, medical details such as the patient's conditions and/or disorders, clinician notes, and the like.

In some embodiments, the EHR 120 may include details about healthcare items (e.g., medications, supplies, mobility aids, and the like) that have been provided to the patient, that have been used to assist or care for the patient, and/or that are recommended to be provided or used for the patient. As another example, the EHR 120 may include information about healthcare services (e.g., assistance services, treatments services, and the like) that have been provided and/or that are recommended to be provided.

In some embodiments, the requesting system 110 is used to generate approval requests to request pre-authorization or approval prior to providing a given service or using supplies. For example, the request prediction system 105 may request approval from one or more other entities (e.g., the patient themselves, family of the patient, an insurer of the patient, and the like) to render the healthcare services. In some embodiments, the requesting system 110 may additionally or alternatively be used to generate approval requests for actions that have already been completed (e.g., to request reimbursement).

In some embodiments, the requesting system 110 may generate approval requests based on the data included in the EHR 120. This may include, for example, automatically generating approval requests (e.g., when the EHR 120 indicates that a service was just rendered and/or that an approval is desired) and/or allowing users to manually generate approval requests. As illustrated, the request system 110 can then transmit the approval requests to the response system 115 and receive responses.

The response system 115 generally corresponds to a computing system used to facilitate evaluation and response to approval requests. For example, the response system 115 may be associated with one or more insurers or other entities responsible for approving and/or paying for healthcare items and services. In some embodiments, the response system 115 can approve or deny (e.g., reject) requests. In some embodiments, the response system 115 may optionally include information about denial reasons (e.g., indicating why the request was denied, such as because it is missing information). The response system 115 may generally respond to requests using any combination of manual and automated evaluations.

In many environments, the particular evaluations used by the response system 115 are highly complex, but also largely (or entirely) unknown to the requesting system 110. This can make it difficult or impossible for the requesting system 110 to know whether a request will be approved. In conventional approaches, therefore, the requesting system 110 may simply transmit requests with minimal evaluation. When a denial is received, the requesting system 110 (or a user associated with the system) may revise the request and re-transmit it. As discussed above, this repetitive process to attempt to secure approval is slow and error-prone. Moreover, as discussed above, this process can incur substantial computational expense. For example, the repeated generation and/or modification of requests, as well as the repeated transmission of the requests and receiving of the responses, consumes substantial computational resources and network bandwidth. This is particular true in batch settings, where the requesting system 110 requests approval for many services at once. These batches can result in significant spikes in network traffic that can persist for a surprisingly long time (due to the repeated denial and re-requests that are common).

In the illustrated system 100, to reduce or eliminate this computational waste and network congestion, the requesting system 110 may interact with the request prediction system 105 to generate output predictions and/or modifications for the approval requests (before sending the requests to the response system 115).

In the illustrated example, the request prediction system 105 includes a model component 125 and a suggestion component 130. Although depicted as discrete components for conceptual clarity, the operations of the depicted components (and others not illustrated) may be combined or distributed across any number of components, and may generally be implemented using hardware, software, or a combination of hardware and software.

The model component 125 generally uses one or more machine learning models to evaluate approval requests provided by the requesting system 110. In some embodiments, the approval requests may specify a set of values for a set of request fields. For example, the request may generated based on a request template having fields such as for information identifying the patient (e.g., by name, address, identifier, and the like), fields for demographic information (e.g., an age field), fields for contextual medical information (e.g., comorbidities, vitals, and the like), fields for the particular service(s) and/or good(s) for which approval is sought (e.g., indicating the services rendered, the medications prescribed, and the like), and any other relevant fields.

In some embodiments, the model component 125 may process some or all of these values using a trained machine learning model (referred to in some embodiments as a claim prediction model, a request prediction model, a request evaluation model, and the like) to generate one or more outputs.

In some embodiments, the outputs include an approval prediction for the request. The approval prediction may generally indicate whether the request will be (or is predicted to be) accepted or approved. For example, the approval prediction may include a binary or categorical prediction (e.g., indicating that the request will be approved, will not be approved, or approval is uncertain) and/or a probability of approval (e.g., a continuous value between 0 and 1 or between 0 and 100, where higher scores indicate higher likelihood of approval).

In some embodiments the outputs include predictions for denial reason(s) that may be used to respond to the request. For example, the model component 125 may generate a probability score for each denial reason of a set of possible denial reasons, and may select one or more of the highest-scored denial reasons for inclusion in the output. In some embodiments, the denial reasons are only provided if the approval prediction indicates a sufficiently high probability of denial. In some embodiments, the denial predictions may be provided regardless of the approval prediction. In some embodiments, the probability of a denial reason indicates the probability that the denial reason will be given (not taking into account the probability of denial in the first place). In some embodiments, the probability of a denial reason indicates the probability that the denial reason will be given, assuming that the claim is denied.

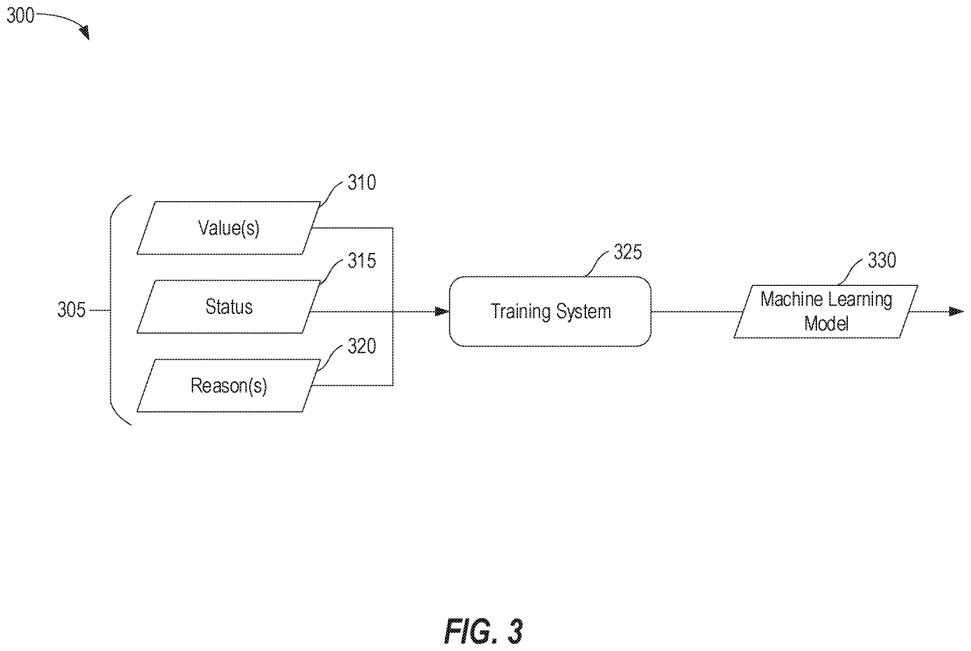

The model component 125 may generally use a variety of machine learning model architectures, depending on the particular implementation. For example, in some embodiments, the machine learning model may comprise a classification model architecture (e.g., regression, a decision tree and/or random forest, and the like). In some embodiments, the model may be trained based on historical request information, as discussed in more detail below with reference to FIG. 3.

The suggestion component 130 may be used to generate suggestions and/or modifications for approval requests. In some embodiments, the suggestion component 130 generates the modifications based at least in part on the output of the model component 125. For example, the suggestion component 130 may evaluate the denial reasons predicted by the model in order to generate suggested modifications. For example, if the predicted denial reason indicates missing data (e.g., a field without an entry), the suggestion component 130 may suggest that this field should be completed.

In some embodiments, in addition to or instead of generating suggestions for user action, the suggestion component 130 may generate and/or implement modifications automatically. For example, based on the predicted denial reason(s), the suggestion component 130 may evaluate the approval request itself and/or the patient's other EHR data 120 to generate an appropriate modification. As one example, if the denial reasons indicate that the claim will be rejected because one or more values are out of the range of values that are typically approved (as learned during training) and/or because two or more fields contain contradictory values, the suggestion component 130 may evaluate the EHR 120 to confirm whether the value(s) included in the approval request are, in fact, correct. If not, the suggestion component 130 may generate a modification including the correct value(s). The suggestion component 130 may then automatically modify the request accordingly, or may provide an output allowing the user to accept or implement the modification (e.g., by clicking a button, as discussed in more detail below).

In some embodiments, the request prediction system 105 and/or the requesting system 110 may also generate overall or aggregated information about the approval requests, as discussed in more detail below. For example, the request prediction system 105 may provide a list of recent, pending, and/or future requests, along with the predicted approval probability and/or denial reasons for each, allowing effective review. In some embodiments, the request prediction system 105 can similarly provide statistics, such as the percentage of requests that are approved for the facility, trends in the approval, and the like.

### Example Workflow for Using Machine Learning Models to Predict Approval and Refine Requests

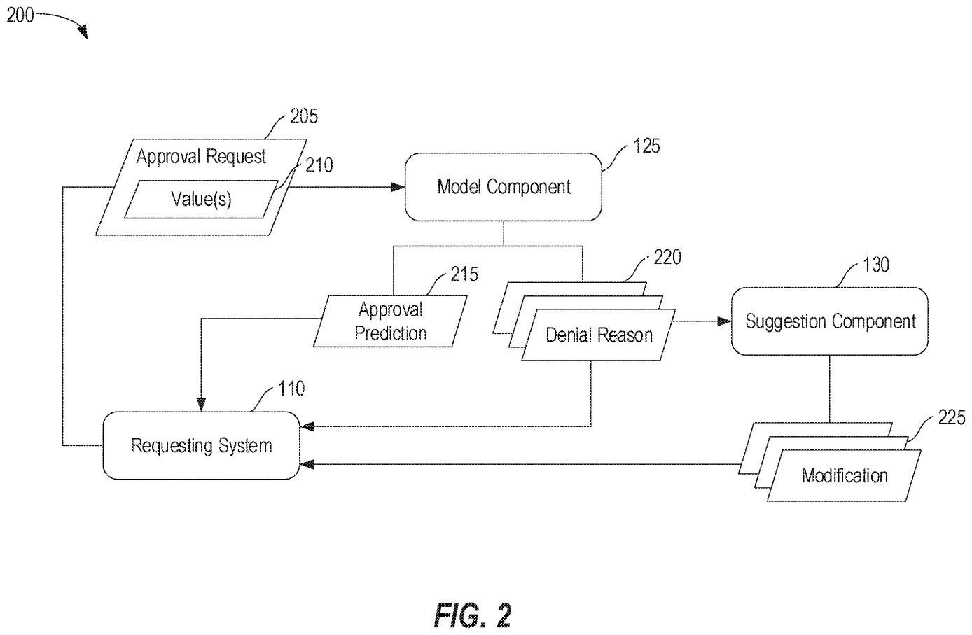

FIG. 2 depicts an example workflow 200 for using machine learning models to predict approval and refine requests, according to some embodiments of the present disclosure. In some embodiments, the workflow 200 is performed by a request prediction system, such as the request prediction system 105 of FIG. 1.

As illustrated, an approval request 205 including or specifying a set of values 210 may be accessed by the model component 125. As used herein, “accessing” data may generally include receiving, requesting, retrieving, collecting, generating, obtaining, or otherwise gaining access to the data. For example, as discussed above, the model component 125 may receive the approval request 205 from another entity or application (e.g., the requesting system 110 of FIG. 1) and/or may retrieve the approval request 205 from a storage repository of such requests.

As discussed above, the approval request may generally correspond to a request for approval and/or reimbursement for one or more healthcare-related goods and/or services that have been provided or are desired (e.g., that are recommended by a clinician). The values 210 generally include the relevant context used to determine whether to accept the request, such as the patient's demographic information, the conditions of the patient that may be relevant to the treatment decisions, the specific treatment or items for which approval is sought, and the like. For example, as discussed above, the approval request 205 may identify the patient, and specify that approval is desired for reimbursement for a treatment action that was performed for the patient, as well as details about why the treatment was provided (e.g., what condition was being treated, the state of the condition such as the stage or other value, and the like).

As illustrated, the model component 125 evaluates the approval request 125 using one or more machine learning models to generate an approval prediction 215 and/or a set of one or more predicted denial reasons 220. That is, the model component 125 may extract and process one or more of the values 210 as input to one or more machine learning models (e.g., the values relevant to claim approval/denial, such as the patient's conditions and state, while refraining from processing irrelevant information such as the patient's name).

In some embodiments, the model component 125 may evaluate other contextual information, such as the identity of the responding entity (e.g., the particular payer to which the request will be transmitted). In some embodiments, a separate machine learning model may be trained for each such entity, allowing the model component 125 to learn to predict insurer-specific models for improved accuracy. In some embodiments, the identity of the respondent can be included as an input to the model itself.

In some embodiments, in addition to or instead of using the responding entity as input, the model component 125 may use the responding entity to identify a corresponding set of payer-specific rules. This may allow the model component 125 to evaluate the approval request 205 using the rule(s) prior to processing the values 210 using the model(s). In some embodiments, this may reduce the computational expense of the request evaluation process. For example, prior to consuming resources using machine learning to evaluate the approval request 205, the model component 125 may first perform a lightweight rules-based analysis to determine whether the request violates any rule(s) used to evaluate requests. If so, the model component 125 may refrain from using the machine learning model(s), and may immediately generate an approval prediction 215 and/or denial reason 220 (e.g., indicating the rule(s) that are violated). If the approval request 205 satisfies the rule(s), the model component 125 may determine that further analysis using the trained machine learning model(s) is warranted. That is, rather than immediately returning an indication that the request will be approved, the model component 125 may adaptively perform further analysis using machine learning to generate a more refined prediction.

In some embodiments, the model component 125 may additionally or alternatively evaluate the approval request 205 using the applicable rule(s) after processing the values 210 using machine learning. For example, if the approval prediction 215 indicates that approval is not likely, the model component 125 may use the rule(s) to determine (or confirm) likely denial reasons 220 (e.g., based on which rule(s) are violated). In some embodiments, by performing this rules-based evaluation only if the approval prediction 215 fails to satisfy defined criteria (e.g., if the probability of approval is below a threshold), the model component 125 can reduce computational expense of the evaluation system (e.g., refraining from using the rules to generate or verify the predicted denial reasons 220 when doing so is not warranted).

As discussed above, the approval prediction 215 may generally indicate whether the approval request 205 is predicted to be accepted or approved. For example, the approval prediction 215 may be a categorical prediction, and/or may indicate the predicted probability that the approval request 205 will be approved (e.g., as a percentage). In the illustrated example, this approval prediction 215 can then be provided (e.g., transmitted) to the requesting system 110. As discussed in more detail below, the requesting system 110 may take various actions in response to the approval prediction 215, such as submitting the approval request 205 to the response system, modifying the approval request 205, and the like.

In the illustrated example, the denial reasons 220 generally indicate the predicted reason(s) that the approval request 205 will be denied (if it is, in fact, denied). For example, in some embodiments, the model(s) may be trained to generate a score for each alternative denial reason of a set of denial reasons (e.g., denial codes specified by the responding entity), where each score indicates the probability that the given denial code is applicable or relevant. In some embodiments, the model component 125 may select the top N denial reasons 220 (e.g., the highest scored reason, or the two highest scored reasons). In some embodiments, the model component 125 may select any denial reasons 220 having a score exceeding a threshold (e.g., higher than 50%). In the illustrated example, the denial reasons 220 are provided to the suggestion component 130, as well as to the requesting system 110, as discussed in more detail below.

In the workflow 200, the suggestion component 130 can evaluate the denial reasons 220 to generate corresponding modifications 225 for the approval request 205. In some embodiments, the suggestion component 130 generates or suggests modifications 225 to obviate the denials. For example, if the denial reason 220 indicates that one or more fields are missing values 210, the suggestion component 130 may suggest that these fields be completed prior to transmitting the approval request 205 for review. In some embodiments, if the suggestion component 130 has access to the patient's EHR data, the modifications 225 may themselves include the relevant values to be added to the approval request 205.

In the illustrated workflow 200, the modifications 225 can then be returned to the requesting system 110. As discussed in more detail below, the requesting system 110 can take a variety of actions, such as outputting the approval prediction 215, denial reasons(s) 220, and/or modifications 225 via a graphical user interface (GUI) for user review. In some embodiments, the user may accept one or more of the modifications 225, causing the system to automatically implement the modification (e.g., updating one or more values 210 of the approval request 205). In some embodiments, the user may implement the modifications 225 (e.g., determining the corrected values 210 and entering them in the approval request 205).

In the illustrated example, the revised approval request 205 can then be provided to the model component 125 for another round of evaluation (e.g., to determine what affect, if any, the modifications have on the approval prediction 215 and/or denial reasons 220). In this way, the requesting system 110 can evaluate and update the approval request 205 prior to submitting it for approval, reducing the computational expense and bandwidth consumed by attempting to secure malformed requests.

### Example Workflow for Training Machine Learning Models to Predict Approval and Refine Requests

FIG. 3 depicts an example workflow 300 for training machine learning models to predict approval and refine requests, according to some embodiments of the present disclosure.

In the illustrated workflow 300, a training system 325 accesses exemplars 305 to train a machine learning model 330. Although depicted as a discrete computing system for conceptual clarity, in some embodiments, the operations of the training system 325 may be combined or distributed across any number of systems, and may generally be implemented using hardware, software, or a combination of hardware and software. In some embodiments, the training system 325 and the request prediction system 105 of FIG. 1 may be implemented as a single system. That is, one computing system may train the machine learning model 330, as well as use the trained machine learning model 330 for runtime predictions. In some embodiments, the training system 325 may be a discrete system, and the machine learning model 330 may be deployed to the request prediction system after training.

In some embodiments, the exemplars 305 are generated based on historical claim information, such as from prior approval requests that were submitted and either accepted or rejected by the responding system.

In the illustrated example, each training exemplar 305 comprises one or more elements of data used as input to the model, as well as one or more targets or labels for the model output. In the workflow 300, the input portion of the exemplar 305 includes one or more values 310 for an approval request. For example, the values 310 may correspond to the values 210 of the approval request 205, discussed above with reference to FIG. 2. Further, as illustrated, the output or label portion of the exemplar 305 include a status 315 (e.g., indicating whether the request was approved or denied). In some embodiments, the label further includes a set of reason(s) 320 (e.g., denial reasons) provided by the responding entity (if the request was denied).

The training system 325 may generally use a variety of operations to train the machine learning model 330, depending on the particular architecture and implementation. For example, in some embodiments, the training system 325 may process the input portion of the exemplar 305 (e.g., the values 310) using the model to generate one or more output predictions (e.g., the approval prediction 215 and/or the predicted denial reasons 220, each of FIG. 2). These predictions can then be compared against the label(s) of the exemplar 305 (e.g., the status 315 and/or reasons 320) to generate a loss (e.g., cross-entropy loss), which may then be used to refine the parameters of the machine learning model 330 (e.g., using backpropagation).

In some embodiments, the machine learning model 330 can be initialized using randomized parameters. As a result, the initial predictions (e.g., at an early training stage) may be relatively random and unreliable. However, by using exemplars 305 to refine the parameters, the model learns to generate more accurate predictions that reflect the patterns observed in the training exemplars 305.

In some embodiments, the training system 325 can train the model using a batch training approach (e.g., batch gradient descent), where multiple exemplars 305 are used to refine the parameters at once. In some embodiments, the training system 325 may use stochastic gradient (e.g., updating the parameters based on each individual exemplar 305).

In some embodiments, the training system 325 may train the machine learning model 330 and deploy the trained model for runtime use, where the parameters of the model are frozen during runtime. In some embodiments, the training system 325 may perform continuous learning after deployment. For example, as requests are processed during runtime (e.g., as approvals and/or denials are received from the responding system(s)), the training system 325 may use these actual responses as labels to generate new exemplars for the runtime requests. These new exemplars can then be used to continue to refine the machine learning model 330, as discussed above.

After training, the training system 325 may deploy the machine learning model 330 to one or more remote and/or local computing systems. Generally, deploying the model may include any operations used to prepare or provide the model for transmission and/or use. For example, the training system 325 may serialize the model parameters, store the parameters locally, initialize the parameters in memory, transmit the parameters to another system, and the like.

### Example System for Machine Learning-Based Patient Intake

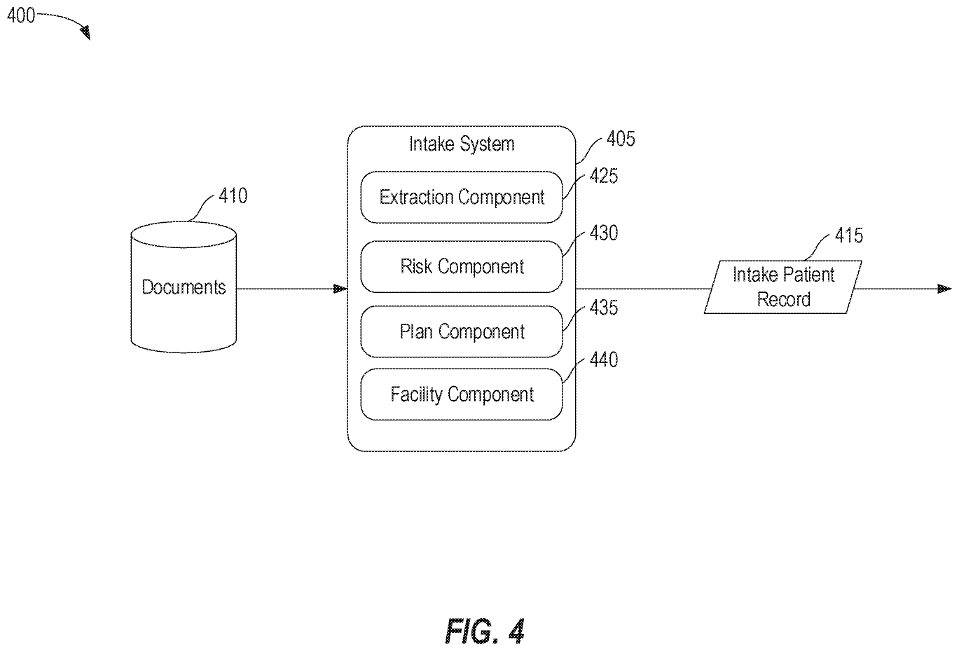

FIG. 4 depicts an example system 400 for machine learning-based patient intake, according to some embodiments of the present disclosure.

In the illustrated example, an intake system 405 accesses documents 410 to generate intake patient records 415. Although depicted as a discrete computing system for conceptual clarity, in some embodiments, the operations of the intake system 405 may be combined or distributed across any number of systems, and may generally be implemented using hardware, software, or a combination of hardware and software. In some embodiments, the intake system 405, the training system 325 of FIG. 3, and/or the request prediction system 105 of FIG. 1 may be implemented as a single system.

As discussed above, the documents 410 may generally include healthcare and/or medical information for a patient that is being considered or evaluated for intake to a facility or ecosystem (e.g., a collection of facilities). This may include, for example, transfer from one facility to another facility. In some embodiments, the documents 410 may be referred to as “raw documents” to indicate that some or all of the documents 410 are not digitized and/or are not formatted (or may be formatted according to an unknown or unverified format). For example, as discussed above, some or all of the documents 410 may be physical documents (e.g., printed paper), or may be scanned versions of such physical documents (e.g., generated using a scanner and/or fax machine). For example, when a patient transfers to a new facility, one or more of the patient's previous facilities may provide the documents 410 to allow the new facility to understand the patient's healthcare journey and status.

The intake patient record 415 generally represents a structured and unified record including the relevant data for the patient in an easy-to-ready and storage efficient format. For example, rather than comprising a large collection of textual documents, the intake patient record 415 may use a well-defined data structure to organize the data. The intake patient record 415 may generally be used to perform or facilitate a wide variety of downstream processes, as discussed below in more detail.

In the illustrated example, the intake system 405 includes an extraction component 425, a risk component 430, a plan component 435, and a facility component 440. Although depicted as discrete components for conceptual clarity, in some embodiments, the operations of the depicted components (and others not illustrated) may be combined or distributed across any number of components and systems.

The extraction component 425 may generally be used to perform an initial extraction of information from the documents 410. For example, in some embodiments, the extraction component 425 may first perform OCR or other techniques to identify and extract textual information from the documents 410. In some embodiments, the extraction component 425 may perform preprocessing on this textual data. For example, in some embodiments, the extraction component 425 may process some or all of the text from a given document 410 using one or more machine learning models to identify or classify what the document 410 is (e.g., whether it is a medication form, a clinician notes form, and the like). In some embodiments, based on the determined document type, the extraction component 425 may extract relevant medical information. For example, if the extraction component 425 determines that the document is a prescription record, the extraction component 425 may extract information such as the medication(s) being prescribed, the dosage, the timing, and the like.

In some embodiments, the extraction component 425 may similarly determine a context of each document 410, such as the date of the document, the author of the document, and the like.

In some embodiments, the extraction component 425 may use the extracted medical information to generate an initial patient record for the patient. For example, the extraction component 425 may populate fields such as the patient's demographics, conditions, medications, and the like. In some embodiments, as part of the record creation, the extraction component 425 may perform various preprocessing operations such as deduplication (e.g., deleting, discarding, or refraining from processing duplicative information). For example, if the documents 410 include two or more documents indicating that the user is currently taking a given medication, the extraction component 425 may refrain from generating multiple such indications of this medication, instead adding a single field in the patient record to indicate that the patient takes the medication. As another example, the extraction component 425 may identify outdated data (e.g., data that has been superseded by more recent documents 410), and may ensure that the patient record either excludes the outdated data, or at least indicates clearly that the data is no longer applicable to the current state of the patient. In some embodiments, some or all of the extraction and/or preprocessing operations performed by the extraction component 425 may be referred to collectively as an automated document ingestion operation.

In some embodiments, the risk component 430 may evaluate this initial patient record using one or more risk prediction machine learning models to generate one or more risk predictions for the patient. In some embodiments, a variety of risk predictions may be configured in a plug-and-play manner. That is, risk prediction models may be trained by any entity (e.g., the training system 325 of FIG. 3) to predict a wide variety of risks, and the models may be used by the risk component 430 to generate updated risk predictions for new patients.

For example, when a new type of risk model is available (e.g., to predict a new risk that the risk component 430 was not previously able to predict), the risk component 430 may begin using this new model to generate subsequent risk predictions for new intake operations, such that the new intake patient records 415 reflect this new risk. As another example, when an updated version of a risk model becomes available, the risk component 430 may begin using this updated model to generate the subsequent risk predictions, ensuring that the newly generated intake patient records 415 are as accurate as possible.

Generally, the risk component 430 may predict a wide variety of risks by processing some or all of the initial patient record using a similarly wide variety of machine learning models. For example, in some embodiments, the risk component 430 may generate risk predictions such as fall risks (e.g., indicating the probability that the patient will suffer a fall), depression risk (e.g., indicating the probability that the patient will suffer from depression), hospitalization risk (e.g., indicating the probability that the patient will be hospitalized), and the like. In some embodiments, the risk predictions generated by the risk component 430 are added to the patient record.

In the illustrated example, the plan component 435 may use one or more machine learning models to evaluate the updated patient record (e.g., the initial record generated by the extraction component 425 and/or the risk prediction(s) generated by the risk component 430) in order to generate and/or score care plans for the patient. For example, the plan component 435 may score input care plans in view of the patient profile to predict how successful the care plan will be in achieving a stated goal, such as reduction in disease progression or symptoms, healing of an injury, and the like. Generally, the care plan may indicate any components of care, including suggested medications, treatment procedures, expected treatment duration, and the like. In some embodiments, the care plan generated by the plan component 435 can be added to the patient record.

In the illustrated system 400, the facility component 440 may evaluate the updated patient record (e.g., the initial record generated by the extraction component 425, the risk prediction(s) generated by the risk component 430, and/or the care plan generated by the plan component 435) in order to generate and/or score care facilities for the patient. In some embodiments, the facility component 440 may use rules-based scoring and/or machine learning-based scoring. For example, a rules-based approach may be used to perform initial filtering of alternative facilities (e.g., to identify facilities that are capable of implementing the care plan), and then machine learning models may be used to score the facilities in order to identify the optimal one.

In some embodiments, the facility component 440 may score input facilities in view of the patient profile to predict how successful the each facility will be in implementing the care plan to achieve a stated goal, such as reduction in disease progression or symptoms, healing of an injury, and the like. For example, such models may be trained based on historical facility information indicating patient characteristics of prior patients, as well as the resulting outcomes experienced by the patients. In some embodiments, the facility scores and/or selection generated by the facility component 440 can be added to the patient record.

In the illustrated example, the intake patient record 415 may include some or all of these generated predictions or data. For example, the intake patient record 415 may include the medical information extracted, from the documents 410, by the extraction component 425, the risk prediction(s) generated by the risk component 430, the care plan(s) generated by the plan component 435, and/or the facility selection generated by the facility component 440.

In some embodiments, the intake patient record 415 can then be used for a variety of tasks, including facilitating intake of the patient to the identified facility (e.g., transmitting the intake patient record 415 to the facility), treatment of the patient at the facility (e.g., using the generated care plan), and/or discharge of the patient from their current facility (for transfer to another facility and/or discharge to home, depending on the contents of the generated care plan).

In these ways, the intake process may be substantially streamlined and improved, resulting in reduced computational expense, reduced latency, and improved patient outcomes

### Example Workflow for Using Machine Learning Models to Facilitate Patient Intake

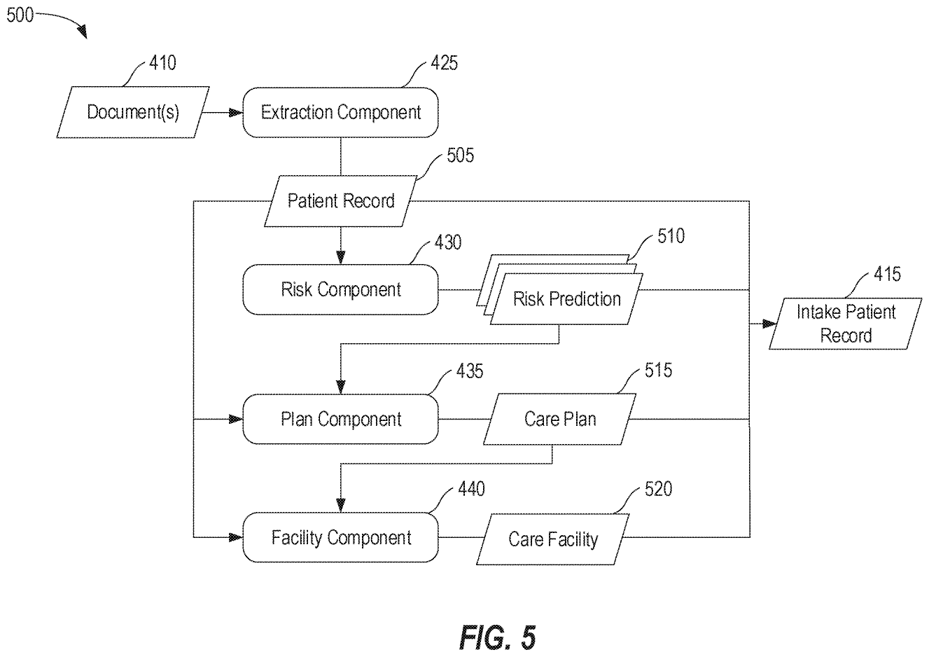

FIG. 5 depicts an example workflow 500 for using machine learning models to facilitate patient intake, according to some embodiments of the present disclosure. In some embodiments, the workflow 500 is performed by an intake system, such as the intake system 405 of FIG. 4.

In the illustrated example, the documents 410 (discussed above with reference to FIG. 4) are accessed by the extraction component 425 to generate a corresponding patient record 505. As discussed above, the documents 410 generally include medical information for a patient, where the information may be entirely unstructured (e.g., natural language text) and/or structured or formatted according to any format (including unknown formats). The extraction component 425 may generally extract the text from the documents 410 and perform various preprocessing operations, as discussed above, to generate the patient record 505.

For example, the extraction component 425 may determine relevant values in the documents 410 based on classifying each document and extracting corresponding information therefrom based on the classification (e.g., where each classification may be associated with an indicated set of fields that should be extracted, such as using rules or regular expressions to specify the values). The extraction component 425 may then clean, de-duplicate, and otherwise prepare the data to generate the patient record 505.

As illustrated, the patient record 505 can then be used as input to the risk component 430. The risk component 430 generates a set of one or more risk predictions 510 based on the patient record 505. For example, as discussed above, the risk component 430 may use one or more machine learning models (e.g., risk prediction models) trained to predict the probability of various adverse events for the patient, such as the fall risk, disease risk, and the like. Generally, the risk component 430 may use a variety models trained for a variety of risks, where each model may consume all or part of the patient record 505 to generate the corresponding risk prediction 510.

In the depicted workflow 500, the patient record 505 and the risk prediction(s) 510 are then accessed by the plan component 435. As discussed above, the plan component 435 may evaluate the patient record 505 and the risk predictions 510 to generate a care plan 515. For example, if the risk predictions 510 indicate high probability of a fall, the plan component 435 may generate a care plan 515 including mobility assistance such as a walker or cane. In some embodiments, the plan component 435 can use a rules-based approach (e.g., where one or more rules or guidelines may specify components of the care plan 515 based on values in the patient record 505 and/or risk predictions 510), a machine learning-based approach (e.g., where one or more machine learning models are used to predict the efficacy of a given care plan (or component thereof) based on the patient record 505 and/or risk predictions 510), and/or a combination of rules-based and machine learning-based approaches.

As illustrated, the care plan 515 and the patient record 505 are then accessed by a facility component 440. Although not depicted in the illustrated example, in some embodiments, the risk prediction(s) 510 may also be accessed by the facility component 440 (either directly, or as part of the patient record 505 if the patient record 505 is updated after generation of the risk predictions 510). The facility component 440 evaluates the patient record 505, risk predictions 510, and/or care plan 515 to generate a selection of a care facility for treatment of the patient.

In some embodiments, as discussed above, the facility component 440 can use a rules-based approach (e.g., where one or more rules or guidelines may specify facilities suitable for implementation of the care plan 515 and/or treatment to mitigate the risk predictions 510), a machine learning-based approach (e.g., where one or more machine learning models are used to predict the outcomes that will be achieved by each facility based on the patient record 505, risk predictions 510, and/or care plan 515), and/or a combination of rules-based and machine learning-based approaches.

In the illustrated workflow 500, the patient record 505 can then be augmented or supplemented using the risk predictions 510, the care plan 515, and/or the care facility 520 to generate a unified intake patient record 415. As discussed above, this intake patient record 415 can then be used to implement or facilitate a wide variety of downstream processes, depending on the particular implementation.

### Example Graphical User Interface for Machine Learning-Based Request Evaluation

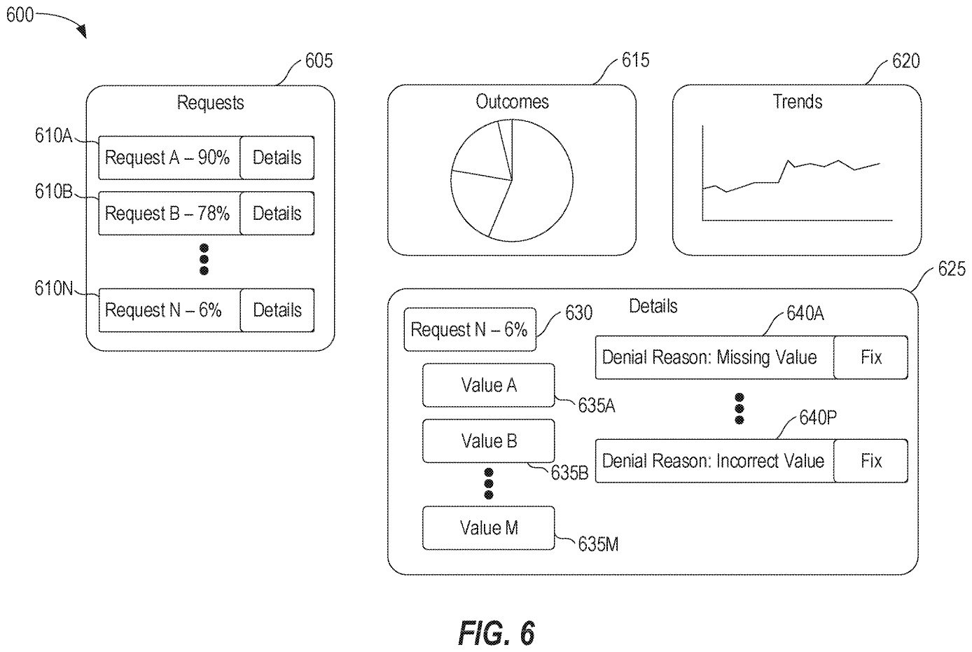

FIG. 6 depicts an example GUI 600 for machine learning-based request evaluation, according to some embodiments of the present disclosure. In some embodiments, the GUI 600 is generated and/or used by a request prediction system, such as the request prediction system 105 of FIG. 1, and/or by an intake system, such as the intake system 405 of FIG. 4.

In the illustrated example, the GUI 600 includes a variety of portions 605, 615, 620, and 625 for illustration and interaction with various aspects of the prediction systems. The illustrated GUI 600 and portions 605, 615, 620, and 625 are depicted for conceptual clarity, and the particular arrangement and contents of each may vary depending on the particular implementation.

In the GUI 600, a portion 605 is used to provide an overview of approval requests (e.g., the approval request 205 of FIG. 2) for the healthcare facility. As discussed above, this may include a list of currently pending requests (e.g., requests which are complete and ready for submission), recently submitted requests (e.g., requests that were recently transmitted to the responding entity, but for which responses may not have been received), and/or expected future requests (e.g., requests which are currently being generated or prepared, and are not yet ready).

In the illustrated example, for each request, a corresponding entry 610A-N is displayed in the portion 605. Each entry 610 may generally include various relevant information for the corresponding request, such as a name of the request, the name of the patient, the name of the respondent to which the request will be sent, the item(s) for which approval or reimbursement is sought, and the like. In some embodiments, a user may select a given entry 610 (e.g., by clicking or hovering over the entry) to see additional details (e.g., to see the full list of values for the request, such as the values 210 of FIG. 2) and/or to edit or modify the request.

In the illustrated example, each entry 610 also indicates the predicted probability of approval (e.g., generated by the request prediction system) for the corresponding request. That is, the entries 610 may include the approval prediction 215 of FIG. 2. In the illustrated example, the approval predictions are numerical percentages, where higher values indicate higher probability of approval. For example, in the illustrated GUI 600, the request corresponding to the entry 610A has a 90% probability of approval, the request corresponding to the entry 610B has a 78% probability of approval, and the request corresponding to the entry 610N has a 6% probability of approval.

Although not depicted in the illustrated example, in some embodiments, all or a portion of each entry 610 may be modified to visualize the corresponding approval probability (e.g., setting the color of each entry 610 based on the probability, such as using a green to red spectrum). Although the illustrated example, depicts numerical approval predictions, in some embodiments, the portion 605 may additionally or alternatively depict categorical predictions, as discussed above.

In some embodiments, the portion 605 may allow the user to sort and/or filter the set of approval requests. For example, the user may sort and/or filter the list of entries 610 based on the corresponding approval predictions (e.g., to only show entries with a sufficiently high or low probability, or to show these entries first). This may allow for rapid review and evaluation of the pending requests.

In the illustrated example, each entry 610 includes an interactive element (e.g., a button) to select the entry (e.g., to request further details). In some embodiments, selecting the “Details” button of a given entry 610 causes the portion 625 of the GUI 600 to be updated based on the detailed information of the corresponding request, as discussed in more detail below.

In the illustrated GUI 600, the portion 615 generally provides information about current and/or prior approval requests associated with the healthcare facility. For example, the illustrated portion 615 includes a pie chart showing the percentages of approval requests that are approved, approved after modification, rejected with no reason given, or rejected with reason given. In some embodiments, the portion 615 may include information for requests from one or more defined windows, such as the last week, the last month, and the like. This can allow the user to quickly visualize how well the facility is performing in terms of request approvals.

Similarly, in the illustrated GUI 600, the portion 620 generally provides information about trends in approval requests associated with the healthcare facility. For example, the illustrated portion 620 includes a line chart showing the percentage of approval requests that are approved over time to illustrate the trend (e.g., increasing, decreasing, or remaining steady). In some embodiments, the portion 620 may include information for requests from one or more defined windows, such as over the last month, the last year, and the like. This can allow the user to quickly visualize how well the facility is performing in terms of request approvals.

In the portion 625, additional information is depicted for a request selected by the user. For example, the user may select the “Details” button on an entry 610 in the portion 605. In the illustrated example, details for the “Request N”, corresponding to the entry 610N, are depicted.

As illustrated, the detailed information can include a name or title of the request in portion 630, as well as fields 635A-M including specific values for the request (e.g., the values 210 of FIG. 2). Specifically, as illustrated, the approval request includes a “Value A,” “Value B,” and “Value M.” As discussed above, these values may include any contextual information (e.g., healthcare or medical information for the patient) used to evaluate the request, and can include numerical values, categorical values, textual strings, and the like.

In the illustrated example, the portion 625 also depicts entries 640A-P for predicted denial reasons (e.g., the denial reasons 220 of FIG. 2) generated by the request prediction system. Specifically, in the illustrated example, the entry 640A specifies the one probable denial reason is one or more missing values, while the entry 640B specifies that another probable denial reason is one or more incorrect values. In some embodiments, as discussed above, the entries 640 may be sorted and/or filtered based on their probabilities (e.g., where the most probable reasons for denial are nearer to the top of the list) and/or based on the impact that correcting each indicated error has on the predicted approval probability (e.g., where corrections that most increase the probability of approval are near the top).

In the illustrated example, each entry 640 further includes an interactive element (e.g., a button labeled “fix”) to implement one or more modifications (e.g., the modifications 225 of FIG. 2) to the approval request. In some embodiments, selecting this interactive element assists the user implementing the modification. For example, the system may open a dialog box allowing the user to enter an updated value for one or more fields. In some embodiments, the system may further retrieve and display relevant medical data (e.g., EHR) for the patient, such as one or more aspects of the patient's data that may contain the correct information for the request.

In some embodiments, as discussed above, selecting this interactive element can additionally or alternatively cause the system to automatically implement the modification. For example, as discussed above, the system may modify the approval request based on the patient's medical data.

In the some embodiments, when an approval request is modified (e.g., by the user or by the system automatically), the system may automatically generate a revised approval prediction (e.g., processing the updated request using the machine learning model(s)). The GUI 600 may then be updated to show the effect of the modification (e.g., updating the corresponding entry 610 in the portion 605 and/or the portion 625).

### Example Method for Using Machine Learning to Evaluate Approval Requests

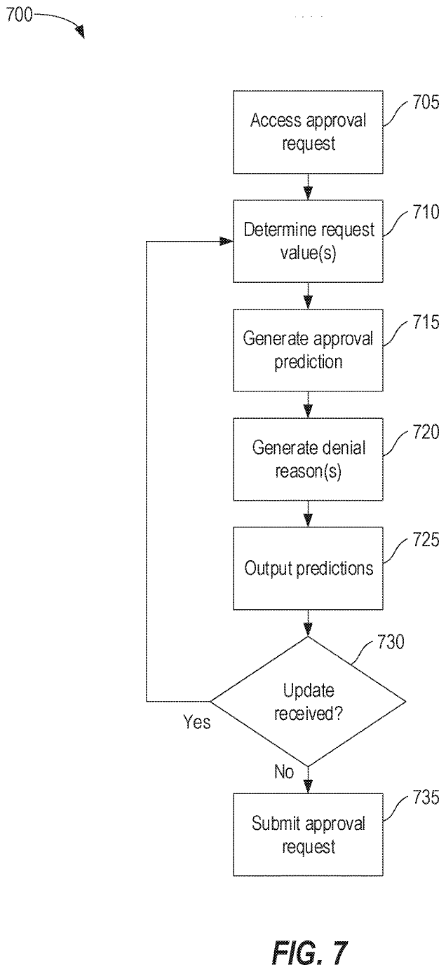

FIG. 7 is a flow diagram depicting an example method 700 for using machine learning to evaluate approval requests, according to some embodiments of the present disclosure. In some embodiments, the method 700 is performed by a request prediction system, such as the request prediction system 105 of FIG. 1.

At block 705, the request prediction system accesses an approval request (e.g., the approval request 205 of FIG. 2). As discussed above, the approval request generally corresponds to a request for approval to perform (or for reimbursement of) a healthcare service and/or provide one or more healthcare items. For example, the approval request may be intended for the insurer of a patient in a facility. In some embodiments, the approval request indicates a set of values (which may include numerical values, textual values, and the like) that provide relevant context for the request (e.g., indicating why the services and/or items are warranted), where the recipient of the request may evaluate these values to determine whether to approve or reject the request.

At block 710, the request prediction system determines the set of request values (e.g., the values 210 of FIG. 2) specified by the request. As discussed above, these values may be included in the request itself, or may be stored in one or more other repositories (e.g., where the request includes links or pointers to one or more of the values). For example, as discussed above, the values may indicate information such as the age of the patient, the service(s) that were provided, the vitals and/or lab results that caused the provider to perform the services, and the like.

At block 715, the request prediction system generates an approval prediction (e.g., the approval prediction 215 of FIG. 2) for the request using one or more trained machine learning models (e.g., using the model component 125 of FIGS. 1-2). In some embodiments, the request prediction system processes some or all of the values as input to the machine learning model to generate the output prediction. As discussed above, the approval prediction generally comprises a prediction as to whether the approval request will be approved by the recipient (e.g., the insurer). For example, the approval prediction may include a binary or categorical prediction (e.g., predicted to approve or predicted to reject), a probability score (e.g., a value between zero and one), and the like.

In some embodiments, as discussed above, the identity of the intended recipient of the approval request can be used as an additional input to the model, allowing the model to generate the approval prediction with respect to the specific recipient (e.g., the payer) that will be evaluating the request. In some embodiments, the request prediction system may use recipient-specific models (where each model is trained to generate predictions for a specific request recipient).

At block 720, the request prediction system generates one or more denial reasons (e.g., the denial reasons 220 of FIG. 2) for the request using one or more machine learning models. In some embodiments, the same model may be used to generate both the approval prediction and the denial reasons. In some embodiments, the denial reason(s) generally indicate the most probable reasons why the request will be denied. In some embodiments, as discussed above, the machine learning model may generate a score for each denial reason of a set of possible denial reasons (e.g., rejection codes used by the recipient of the request), and the request prediction system may generate the denial reasons by selecting one or more of the highest-scored reasons.

At block 725, the request prediction system outputs the predictions, such as via one or more GUIs (e.g., the GUI 600 of FIG. 6). In some embodiments, the request prediction system outputs the predictions to the requesting entity (e.g., a user that generated the request and/or is associated with the healthcare provider that provided the goods and/or services). In some embodiments, as discussed above, the predictions may be provided prior to the requestor sending the request to the actual recipient (e.g., the insurer), allowing the requestor to refine or modify the requests prior to transmitting them.

At block 730, the request prediction system determines whether any updated value(s) for the request have been received. For example, as discussed above, after reviewing the approval prediction and/or denial reasons, the user may enter a new value (e.g., changing the previous value of one or more fields, or adding a value to a field that previously did not include a value in the request). In some embodiments, as discussed above, the denial reasons may specify the proposed new values (e.g., generated based on evaluating EHR associated with the patient). These proposed values (e.g., the modifications 225 of FIG. 2) may be entered upon approval of the user, and/or may be automatically used to update the request without user input.

If one or more updates have been received, the method 700 returns to block 710, where the request prediction system can evaluate the updated request to generate new predictions. If no updates have been received (e.g., the user indicated to submit the request without modification), the method 700 continues to block 735. At block 735, the request prediction system (or another system) submits the approval request to the responding system (e.g., the response system 115 of FIG. 1). For example, the request prediction system may transmit the request for review by the intended recipient (e.g., the insurer of the patient).

In these ways, using the method 700, the request prediction system can significantly improve the probability of request approval, reducing or eliminating the latency, network traffic, and computational expense of repeated denial, revision, and resubmission of requests.

### Example Method for Training Machine Learning Models to Evaluate Approval Requests

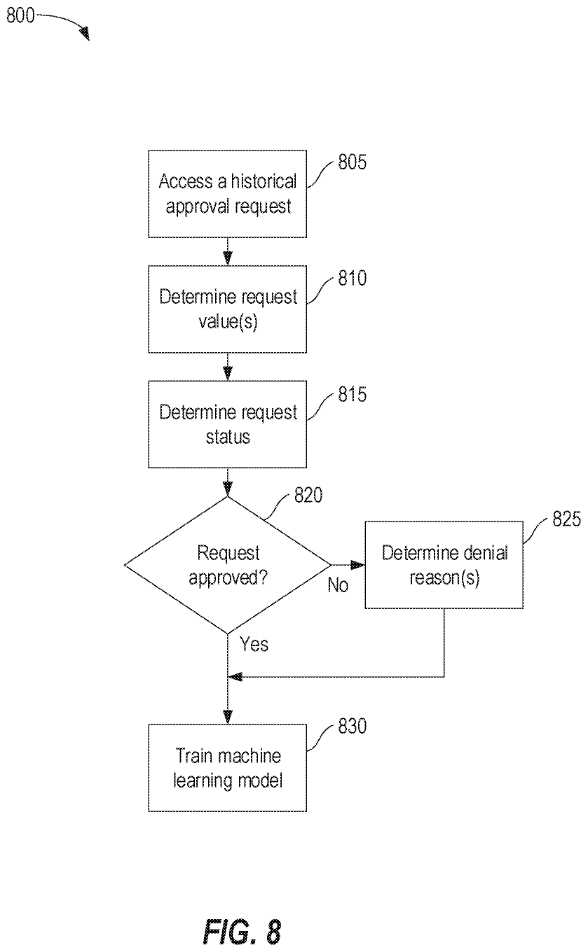

FIG. 8 is a flow diagram depicting an example method 800 for training machine learning models to evaluate approval requests, according to some embodiments of the present disclosure. In some embodiments, the method 700 is performed by a training system, such as the training system 325 of FIG. 3. In some embodiments, as discussed above, the training system may correspond to all or a portion of the request prediction system 105 of FIGS. 1-2.

At block 805, the training system accesses a historical approval request (e.g., the exemplar 305 of FIG. 3). In some embodiments, as discussed above, the historical approval request generally corresponds to a previous request that was submitted (e.g., to an insurer) and for which a response has already been received (e.g., approval or denial).

At block 810, the training system determines the values (e.g., the values 310 of FIG. 3) specified for the historical request. As discussed above, these values may be used as the input to the machine learning model during training.

At block 815, the training system determines the status (e.g., the status 315 of FIG. 3) of the request. For example, as discussed above, the training system may determine whether the request was approved or denied. In some embodiments, this status may be used as a label of the exemplar (e.g., the target output used to train the model).

At block 820, the training system determines whether the request was approved (based on the determined status). If so, the method 800 continues to block 830 discussed in more detail below. If the request was denied, the method 800 continues to block 825.

At block 825, the training system determines the denial reason(s) (e.g., the reasons 320 of FIG. 3), if any are available, for the historical request. For example, as discussed above, the respondent may specify why the request was denied using denial codes (e.g., selecting the reason from a predefined list of possible denial reasons), such as “missing information” for one or more fields, “value does not meet approval criteria” for one or more fields, and the like. In some embodiments, as discussed above, these denial reasons may be used as a label to train the model.

At block 830, the training system trains the machine learning model based on the historical approval request. As discussed above, the particular operations used to train the model may vary depending on the particular implementation. Generally, the values of the request (determined at block 810) are used as input to the model to generate corresponding output predictions (e.g., an approval prediction and one or more denial reason predictions). The labels (e.g., the status determined at block 815 and/or the denial reasons determined at block 825) can then be compared against the model output to generate a loss, which can be used to refine the model parameters (e.g., via backpropagation).

In some embodiments, if the request was approved, the training system may use a placeholder denial reason (e.g., “no denial” or “request approved”) to train the denial reason prediction portion of the model. In this way, the model may learn to predict not only approval, but also to generate a denial reason indicating that the request will not be denied.

In some embodiments, as discussed above, the identity of the responding entity may also be used as input to the model. In some embodiments, the training system may identify the responding entity and train the corresponding model (e.g., training payer-specific models).

Although the illustrated example depicts training the model based on a single historical approval request the method 800 may be repeated any number of times to train the model (e.g., using a large number of historical requests). Further, although the illustrated example depicts updating the model parameters based on a single request (e.g., using stochastic gradient descent), in some embodiments, the training system may train the model based on multiple historical requests at a time (e.g., using batch gradient descent).

### Example Method for On-Line Refinement of Machine Learning Models to Evaluate Approval Requests

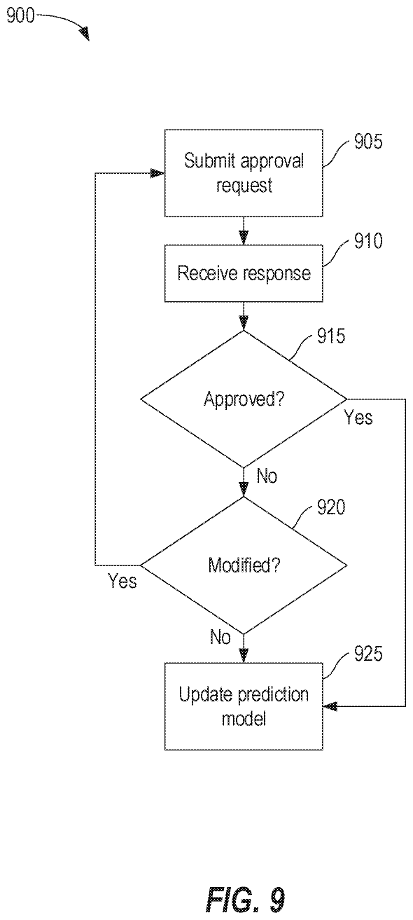

FIG. 9 is a flow diagram depicting an example method 900 for on-line refinement of machine learning models to evaluate approval requests, according to some embodiments of the present disclosure. In some embodiments, the method 900 is performed by a request prediction system, such as the request prediction system 105 of FIG. 1.

At block 905, the request prediction system submits an approval request (e.g., to the response system 115 of FIG. 1). For example, after a user has created and/or approved a request, the request can be submitted to the insurer for review. In some embodiments, as discussed above, the request prediction system may have already evaluated the request to generate prediction(s) prior to the request being submitted to the responder.

At block 910, the request prediction system receives a response from the responding entity. As discussed above, the response may generally include approval denial without specified reasons, denial with one or more specified reasons, and the like.

At block 915, the request prediction system determines whether the request was approved. If so, the method 900 continues to block 925. If not, the method 900 continues to block 920.

At block 920, the request prediction system determines whether the request has been modified (e.g., by a user). For example, as discussed above, if a request is denied, the user may determine to modify it (e.g., changing one or more values or adding one or more values) and resubmit it in an attempt to secure approval. If the request has been modified, the method 900 returns to block 905. If the request has not been modified, the method 900 continues to block 925.

At block 925, the request prediction system updates the prediction model based on the response from the responding entity. For example, as discussed above, the request prediction system may use the request and the corresponding response (along with denial reasons, if available) as a new training exemplar to further finetune or refine the model. This may allow the request prediction system to perform continuous learning, insuring that the predictions remain accurate and allowing the system to dynamically adjust to changing circumstances.

### Example Method for Using Machine Learning to Improve Patient Intake

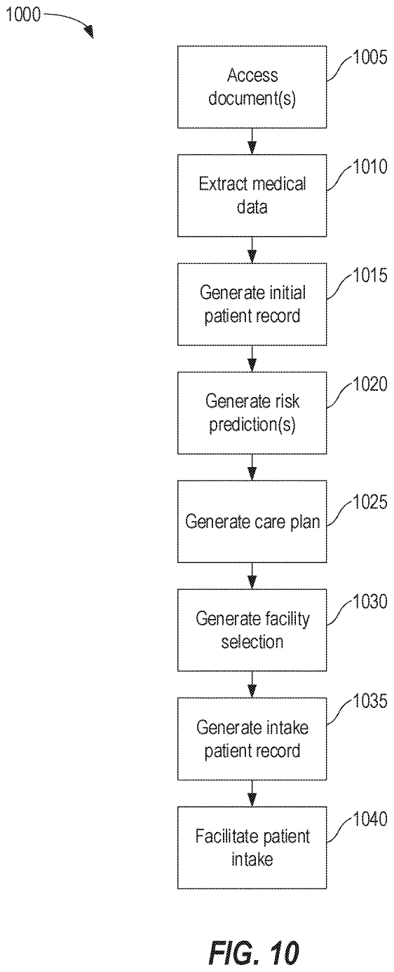

FIG. 10 is a flow diagram depicting an example method 1000 for using machine learning to improve patient intake, according to some embodiments of the present disclosure. In some embodiments, the method 1000 is performed by an intake system, such as the intake system 405 of FIG. 4. In some embodiments, as discussed above, the intake system may correspond to all or a portion of the request prediction system 105 of FIGS. 1-2.

At block 1005, the intake system accesses a set of patient documents (e.g., the documents 410 of FIGS. 4-5) for a patient that is being evaluated (e.g., for intake to a healthcare facility or system). As discussed above, the documents may generally include medical data in any format, such as physical documents, scanned and/or faxed documents, and the like. In some embodiments, the documents are received from the patient's current and/or prior healthcare providers (e.g., to facilitate transfer).

At block 1010, the intake system extracts medical data from the documents. Generally, the particular operations used to extract the medical data may vary depending on the particular implementation. For example, in some embodiments, the intake system may use OCR or other techniques to identify and extract textual information from the documents. This text may then be evaluated using various techniques to identify or classify each document based on document type (e.g., using a trained machine learning model, using keyword matching, and the like). Based on this document type, the relevant medical information can then be extracted.

At block 1015, the intake system generates an initial patient record (e.g., the patient record 505 of FIG. 5) based on the extracted medical data. For example, as discussed above, the intake system may generate a record that includes the relevant medical information such as the patient's demographics, conditions, medications, and the like.

At block 1020, the intake system generates one or more risk predictions (e.g., the risk predictions 510 of FIG. 5) for the patient based on the patient record. For example, the intake system may process some or all of the information contained in the initial patient record using one or more risk prediction machine learning models to generate corresponding predictions with respect to the risks (e.g., a fall risk, a depression risk, and the like). As discussed above, these risk predictions may generally quantify the risk of the corresponding condition, such as the probability that the patient will experience the condition, the predicted intensity or harm caused by the condition, and the like.

At block 1025, the intake system generates a care plan (e.g., the care plan 515 of FIG. 5) based on the initial patient record. In some embodiments, the care plan is generated based further on the risk prediction(s) generated. In some embodiments, as discussed above, the intake system may process some or all of the information in the initial patient record and/or some or all of the risk predictions as input to one or more care plan generation or evaluation machine learning models. For example, as discussed above, the machine learning model may be trained to predict outcomes and/or score proposed care plans based on the particular patient information (e.g., predicting what outcomes the patient will encounter if the indicated care plan is used).

At block 1030, the intake system generates a facility selection (e.g., the care facility 520 of FIG. 5) based on the initial patient record. In some embodiments, the care facility is generated based further on the risk prediction(s) and/or the care plan that has been generated. In some embodiments, as discussed above, the intake system may process some or all of the information in the initial patient record, some or all of the risk predictions, and/or some or all of the care plan as input to one or more facility selection or evaluation machine learning models. For example, as discussed above, the machine learning model may be trained to predict outcomes and/or score proposed facilities based on the particular patient information (e.g., predicting what outcomes the patient will encounter if the patient is treated at the indicated care facility).

At block 1035, the intake system generates an intake patient record (e.g., the intake patient record 415 of FIG. 4) including the information from the initial patient record, the one or more risk predictions, the care plan, and the selected treatment facility. In some embodiments, as discussed above, the intake system may add the generated information to the initial patient record in order to generate a unified intake patient record containing the relevant healthcare information and predictions for the patient. As discussed above, the intake patient record can then be used to facilitate or implement a variety of downstream operations.

For example, at block 1040, the intake system facilitates patient intake based on the intake patient record. As discussed above, this can generally include performing intake of the patient to the selected care facility, performing discharge of the patient from their prior or current facility, transferring the patient to the new facility, treating the patient at the facility (using the generated care plan), monitoring the patient for the identified risk(s), discharging the patient after treatment, and the like.

### Example Method for Evaluating Approval Requests

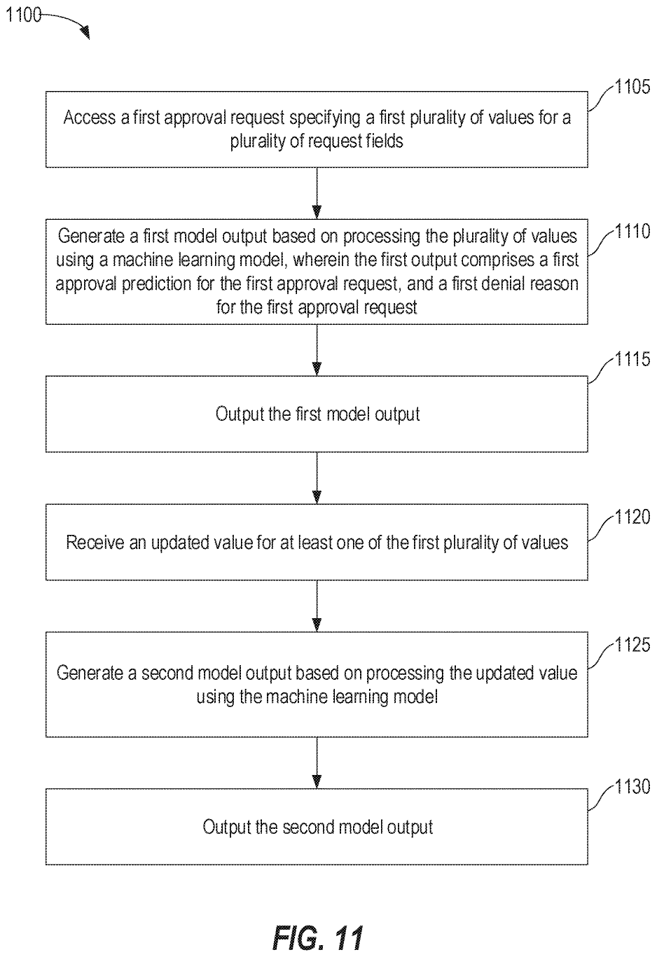

FIG. 11 is a flow diagram depicting an example method 1100 evaluating approval requests, according to some embodiments of the present disclosure. In some embodiments, the method 1100 is performed by a request prediction system, such as the request prediction system 105 of FIG. 1.

At block 1105, a first approval request (e.g., the approval request 205 of FIG. 2) specifying a first plurality of values (e.g., the values 210 of FIG. 2) for a plurality of request fields is accessed.

At block 1110, a first model output is generated based on processing the plurality of values using a machine learning model, wherein the first output comprises a first approval prediction (e.g., the approval prediction 215 of FIG. 2) for the first approval request, and a first denial reason (e.g., the denial reasons 220 of FIG. 2) for the first approval request.

At block 1115, the first model output is outputted.

At block 1120, an updated value is received for at least one of the first plurality of values.

At block 1125, a second model output is generated based on processing the updated value using the machine learning model.

At block 1130, the second model output is outputted.

### Example Method for Patient Intake

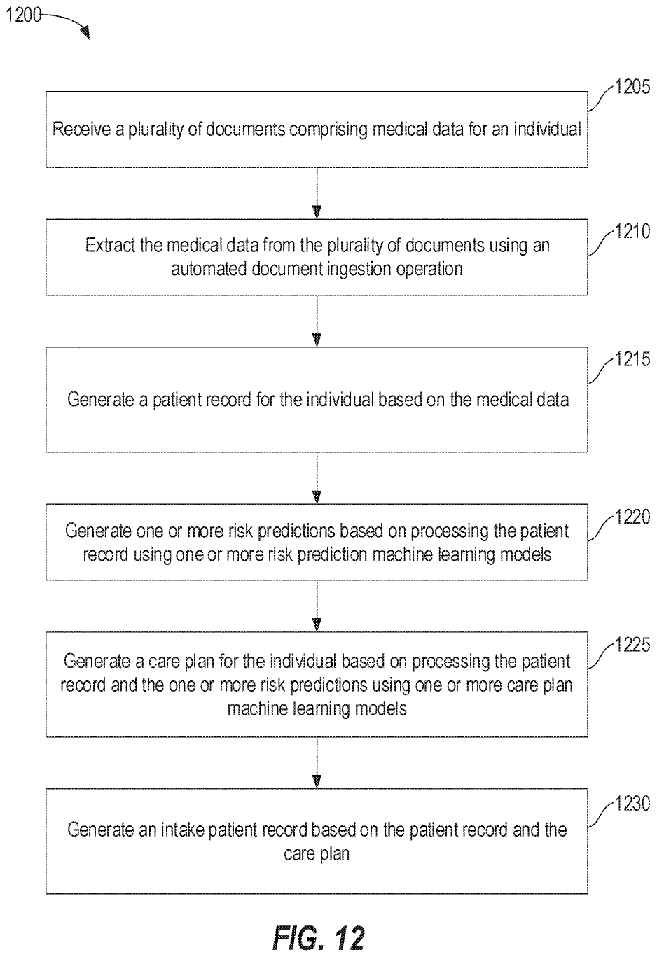

FIG. 12 is a flow diagram depicting an example 1200 method patient intake, according to some embodiments of the present disclosure. In some embodiments, the method 1200 is performed by an intake system, such as the intake system 405 of FIG. 4.

At block 1205, a plurality of documents (e.g., the documents 410 of FIGS. 4-5) comprising medical data for an individual is received.

At block 1210, the medical data is extracted from the plurality of documents using an automated document ingestion operation.

At block 1215, a patient record (e.g., the patient record 505 of FIG. 5) is generated for the individual based on the medical data.

At block 1220, one or more risk predictions (e.g., the risk predictions 510 of FIG. 5) are generated based on processing the patient record using one or more risk prediction machine learning models.

At block 1225, a care plan (e.g., the care plan 515 of FIG. 5) is generated for the individual based on processing the patient record and the one or more risk predictions using one or more care plan machine learning models.

At block 1230, an intake patient record (e.g., the intake patient record 415 of FIGS. 4-5) is generated based on the patient record and the care plan.

### Example Processing System for Request Evaluation Machine Learning

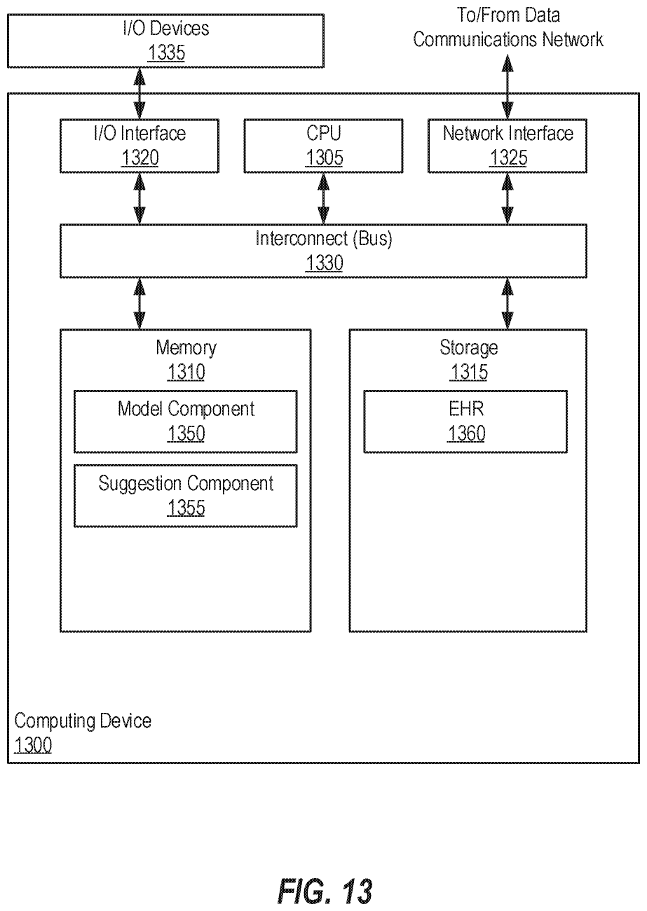

FIG. 13 depicts an example computing device 1300 configured to perform various aspects of the present disclosure, according to some embodiments disclosed herein. Although depicted as a physical device, in embodiments, the computing device 1300 may be implemented using virtual device(s), and/or across a number of devices (e.g., in a cloud environment). In one embodiment, the computing device 1300 corresponds to a request prediction system, such as the request prediction system 105 of FIG. 1.

As illustrated, the computing device 1300 includes a CPU 1305, memory 1310, storage 1315, a network interface 1325, and one or more input/output (I/O) interfaces 1320. In the illustrated embodiment, the CPU 1305 retrieves and executes programming instructions stored in memory 1310, as well as stores and retrieves application data residing in storage 1315. The CPU 1305 is generally representative of a single CPU and/or GPU, multiple CPUs and/or GPUs, a single CPU and/or GPU having multiple processing cores, and the like. The memory 1310 is generally included to be representative of a random access memory. Storage 1315 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1335 (such as keyboards, monitors, etc.) are connected via the I/O interface(s) 1320. Further, via the network interface 1325, the computing device 1300 can be communicatively coupled with one or more other devices and components (e.g., via a network, which may include the Internet, local network(s), and the like). As illustrated, the CPU 1305, memory 1310, storage 1315, network interface(s) 1325, and I/O interface(s) 1320 are communicatively coupled by one or more buses 1330.

In the illustrated embodiment, the memory 1310 includes a model component 1350 and a suggestion component 1355, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1310, in embodiments, the operations of the depicted components (and others not illustrated) may be implemented using hardware, software, or a combination of hardware and software.

In some embodiments, the model component 1350 (which may correspond to the model component 125 of FIGS. 1-2) can be used to generate, train, update, and/or use machine learning models to evaluate approval requests (e.g., the approval request 205 of FIG. 2), as discussed above. For example, the model component 1350 may use machine learning models to generate approval predictions (e.g., the approval prediction 215 of FIG. 2) and/or predicted denial reasons (e.g., the denial reasons 220 of FIG. 2) based on approval requests being created.

In some embodiments, the suggestion component 1355 (which may correspond to the suggestion component 130 of FIGS. 1-2) can be used to generate proposed modifications (e.g., the modifications 225 of FIG. 2) to the approval request in order to improve the probability that the request will be approved. In some embodiments, as discussed above, generating these modifications may include suggesting that the user determine the correct value(s), suggesting corrected value(s) directly, and/or linking or pointing the user to a document or record (e.g., in the EHR 1360) where the correct value(s) may be found.

In the illustrated example, the storage 1315 includes EHR 1360 (which may correspond to the EHR 120 of FIG. 1). Although not depicted in the illustrated example, in some embodiments, the storage 1315 may also include other data such as training data (e.g., prior requests, such as the exemplar 305 of FIG. 3), machine learning models, and the like. Although depicted as residing in storage 1315, the depicted data may be stored in any suitable location, including memory 1310.

Generally, the depicted components (and others not depicted) in memory 1310 may evaluate and/or use the depicted data (and others not depicted) in storage 1315 to provide accurate and efficient request evaluations, as discussed above.

### Example Processing System for Request Evaluation Machine Learning

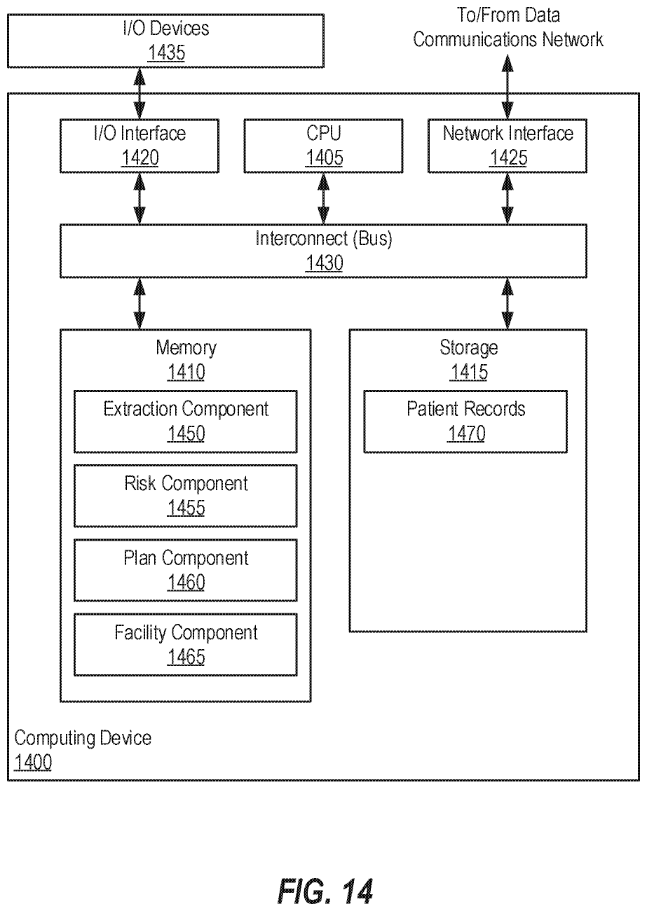

FIG. 14 depicts an example computing device 1400 configured to perform various aspects of the present disclosure, according to some embodiments disclosed herein. Although depicted as a physical device, in embodiments, the computing device 1400 may be implemented using virtual device(s), and/or across a number of devices (e.g., in a cloud environment). In one embodiment, the computing device 1400 corresponds to an intake system, such as the intake system 405 of FIG. 4.

As illustrated, the computing device 1400 includes a CPU 1405, memory 1410, storage 1415, a network interface 1425, and one or more input/output (I/O) interfaces 1420. In the illustrated embodiment, the CPU 1405 retrieves and executes programming instructions stored in memory 1410, as well as stores and retrieves application data residing in storage 1415. The CPU 1405 is generally representative of a single CPU and/or GPU, multiple CPUs and/or GPUs, a single CPU and/or GPU having multiple processing cores, and the like. The memory 1410 is generally included to be representative of a random access memory. Storage 1415 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1435 (such as keyboards, monitors, etc.) are connected via the I/O interface(s) 1420. Further, via the network interface 1425, the computing device 1400 can be communicatively coupled with one or more other devices and components (e.g., via a network, which may include the Internet, local network(s), and the like). As illustrated, the CPU 1405, memory 1410, storage 1415, network interface(s) 1425, and I/O interface(s) 1420 are communicatively coupled by one or more buses 1430.

In the illustrated embodiment, the memory 1410 includes an extraction component 1450, a risk component 1455, a plan component 1460, and a facility component 1465, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1410, in embodiments, the operations of the depicted components (and others not illustrated) may be implemented using hardware, software, or a combination of hardware and software.

In some embodiments, the extraction component 1450 (which may correspond to the extraction component 425 of FIGS. 4-5) can be used to extract medical data from patient documents (e.g., the documents 410 of FIGS. 4-5), as discussed above. For example, the extraction component 1450 may use OCR and/or machine learning to extract text and identify the relevant medical information.

In some embodiments, the risk component 1455 (which may correspond to the risk component 430 of FIGS. 4-5) can be used to generate risk predictions (e.g., the risk predictions 510 of FIG. 5), as discussed above. For example, the risk component 1455 may use machine learning to evaluate one or more features extracted from the documents in order to predict future risk(s) for the patient, such as the probability of the patient obtaining one or more conditions or suffering one or more negative events.

In some embodiments, the plan component 1460 (which may correspond to the plan component 435 of FIGS. 4-5) can be used to generate care plans (e.g., the care plan 515 of FIG. 5), as discussed above. For example, the plan component 1460 may use machine learning to evaluate one or more features extracted from the documents and/or the predicted risks in order to predict outcomes for the patient given various care plans, allowing the system to select or generate an optimized care plan for the patient.

In some embodiments, the facility component 1465 (which may correspond to the facility component 440 of FIGS. 4-5) can be used to generate facility selections (e.g., the care facility 520 of FIG. 5), as discussed above. For example, the facility component 1465 may use machine learning to evaluate one or more features extracted from the documents, the risk predictions, and/or the care plan in order to predict which facility is best suited to care for the patient.

In the illustrated example, the storage 1415 includes patient records 1470 (which may correspond to the initial patient record 505 and/or the intake patient record 415, each of FIG. 5). Although not depicted in the illustrated example, in some embodiments, the storage 1415 may also include other data such as training data, machine learning models, and the like. Although depicted as residing in storage 1415, the depicted data may be stored in any suitable location, including memory 1410.

Generally, the depicted components (and others not depicted) in memory 1410 may evaluate and/or use the depicted data (and others not depicted) in storage 1415 to provide accurate and efficient patient intake, as discussed above.

### Example Clauses

Clause 1: A method, comprising: accessing a first approval request specifying a first plurality of values for a plurality of request fields; generating a first model output based on processing the plurality of values using a machine learning model, wherein the first output comprises: a first approval prediction for the first approval request, and a first denial reason for the first approval request; outputting the first model output; receiving an updated value for at least one of the first plurality of values; generating a second model output based on processing the updated value using the machine learning model; and outputting the second model output.
Clause 2: A method according to Clause 1, wherein: the first approval prediction indicates a probability that the first approval request will be approved, and the first denial reason indicates a most likely reason for denial of the first approval request.
Clause 3: A method according to any of Clauses 1-2, wherein generating the first model output further comprises: generating a plurality of probability scores for a plurality of denial reasons; and selecting the first denial reason based on the plurality of probability scores.
Clause 4: A method according to Clause 3, wherein generating the first model output further comprises selecting at least a second denial reason based on the plurality of probability scores.
Clause 5: A method according to any of Clauses 1-4, further comprising: generating a suggestion including the updated value based on the first denial reason; and outputting the suggestion.
Clause 6: A method according to Clause 5, wherein: outputting the suggestion comprises outputting an interactive element on a graphical user interface (GUI), and receiving the updated value comprises determining that a user selected the interactive element.
Clause 7: A method according to Clause 6, further comprising updating the first approval request based on the updated value.
Clause 8: A method according to any of Clauses 1-7, further comprising: generating a second updated value based on a second denial reason; and automatically updating the first approval request based on the second updated value.
Clause 9: A method, comprising: receiving a plurality of documents comprising medical data for an individual; extracting the medical data from the plurality of documents using an automated document ingestion operation; generating a patient record for the individual based on the medical data; generating one or more risk predictions based on processing the patient record using one or more risk prediction machine learning models; generating a care plan for the individual based on processing the patient record and the one or more risk predictions using one or more care plan machine learning models; and generating an intake patient record based on the patient record and the care plan.
Clause 10: A method according to Clause 9, further comprising selecting a care facility based on processing the intake patient record using a facility machine learning model.
Clause 11: A method according to Clause 10, further comprising facilitating intake of the individual to the care facility based on the intake patient record.
Clause 12: A method according to any of Clauses 10-11, wherein the intake patient record comprises the medical data, the one or more risk predictions, the care plan, and the care facility.
Clause 13: A method according to any of Clauses 9-12, wherein generating the patient record comprises discarding duplicative information extracted from the plurality of documents.
Clause 14: A method according to any of Clauses 9-13, further comprising: accessing a new risk prediction machine learning model; and generating subsequent patient records using the new risk prediction machine learning model.
Clause 15: A method according to any of Clauses 9-14, further comprising outputting the intake patient record via a graphical user interface (GUI).
Clause 16: Any patentable subject matter described herein.
Clause 17: A processing system, comprising: one or more processors; and one or more memories collectively comprising computer-executable instructions which, when executed on any combination of the one or more processors, cause the processing system to perform a method in accordance with any one of Clauses 1-15.
Clause 18: A system, comprising means for performing a method in accordance with any one of Clauses 1-15.
Clause 19: One or more non-transitory computer readable media collectively containing, in any combination, computer program code that, when executed by operation of a computing system, causes the computing system to perform a method in accordance with any one of Clauses 1-15.
Clause 20: A computer program product embodied on a computer-readable storage medium comprising code for performing a method in accordance with any one of Clauses 1-15.

### Additional Considerations

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system) or related data available in the cloud. For example, the response prediction system and/or the intake system could execute on a computing system in the cloud and train and/or use machine learning models to evaluate approval requests and/or facilitate patient intake. In such a case, the system could receive and process the various patient data, and store the models and definitions a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a first approval request specifying a first plurality of values for a plurality of request fields;
generating a first model output based on processing the plurality of values using a machine learning model, wherein the first output comprises:
a first approval prediction for the first approval request, and
a first denial reason for the first approval request;
outputting the first model output;
receiving an updated value for at least one of the first plurality of values;
generating a second model output based on processing the updated value using the machine learning model; and
outputting the second model output.

2. The method of claim 1, wherein:
the first approval prediction indicates a probability that the first approval request will be approved, and
the first denial reason indicates a most likely reason for denial of the first approval request.

3. The method of claim 1, wherein generating the first model output further comprises:
generating a plurality of probability scores for a plurality of denial reasons; and
selecting the first denial reason based on the plurality of probability scores.

4. The method of claim 3, wherein generating the first model output further comprises selecting at least a second denial reason based on the plurality of probability scores.

5. The method of claim 1, further comprising:
generating a suggestion including the updated value based on the first denial reason; and
outputting the suggestion.

6. The method of claim 5, wherein:
outputting the suggestion comprises outputting an interactive element on a graphical user interface (GUI), and
receiving the updated value comprises determining that a user selected the interactive element.

7. The method of claim 6, further comprising updating the first approval request based on the updated value.

8. The method of claim 1, further comprising:
generating a second updated value based on a second denial reason; and
automatically updating the first approval request based on the second updated value.

9. A method, comprising:
receiving a plurality of documents comprising medical data for an individual;
extracting the medical data from the plurality of documents using an automated document ingestion operation;
generating a patient record for the individual based on the medical data;
generating one or more risk predictions based on processing the patient record using one or more risk prediction machine learning models;
generating a care plan for the individual based on processing the patient record and the one or more risk predictions using one or more care plan machine learning models; and
generating an intake patient record based on the patient record and the care plan.

10. The method of claim 9, further comprising selecting a care facility based on processing the intake patient record using a facility machine learning model.

11. The method of claim 10, further comprising facilitating intake of the individual to the care facility based on the intake patient record.

12. The method of claim 10, wherein the intake patient record comprises the medical data, the one or more risk predictions, the care plan, and the care facility.

13. The method of claim 9, wherein generating the patient record comprises discarding duplicative information extracted from the plurality of documents.

14. The method of claim 9, further comprising:
accessing a new risk prediction machine learning model; and
generating subsequent patient records using the new risk prediction machine learning model.

15. The method of claim 9, further comprising outputting the intake patient record via a graphical user interface (GUI).

16. A processing system, comprising:
one or more processors; and
one or more memories collectively comprising computer-executable instructions which, when executed on any combination of the one or more processors, cause the processing system to perform an operation comprising:
accessing a first approval request specifying a first plurality of values for a plurality of request fields;
generating a first model output based on processing the plurality of values using a machine learning model, wherein the first output comprises:
a first approval prediction for the first approval request, and
a first denial reason for the first approval request;
outputting the first model output;
receiving an updated value for at least one of the first plurality of values;
generating a second model output based on processing the updated value using the machine learning model; and
outputting the second model output.

17. The processing system of claim 16, wherein:
the first approval prediction indicates a probability that the first approval request will be approved, and
the first denial reason indicates a most likely reason for denial of the first approval request.

18. The processing system of claim 16, wherein generating the first model output further comprises:
generating a plurality of probability scores for a plurality of denial reasons; and
selecting the first denial reason based on the plurality of probability scores.

19. The processing system of claim 18, wherein generating the first model output further comprises selecting at least a second denial reason based on the plurality of probability scores.

20. The processing system of claim 16, further comprising:
generating a suggestion including the updated value based on the first denial reason; and
outputting the suggestion.
